(19) United States
(12) Patent Application Publication
(10) Pub. No.: US 2014/0194783 A9
(43) Pub. Date: Jul. 10, 2014

(54) System and method for tattoo removal

(71) Applicant: Stephen Feldman, New York, NY (US)

(72) Inventor: Stephen Feldman, New York, NY (US)

(21) Appl. No.: 13/573,624

(22) Filed: Sep. 28, 2012

Related U.S. Application Data
(63) Continuation-in-part of application No. 12/381,134, filed on Mar. 6, 2009.
(60) Provisional application No. 61/068,369, filed on Mar. 7, 2008.

(51) Int. Cl. A61N 7/00

(52) U.S. Cl. 601/2

(57) ABSTRACT

A combination device for applying a treatment of light and ultrasound on a tattooed area of a patient desiring tattoo removal. The device includes an ultrasound and a LED light source. The ultrasound source produces high-frequency ultrasound waves. These waves are applied directly to the tattooed area for a first specified period of time resulting in release of tattoo ink from tattooed cells. The LED source produces a continuous high-intensity light that is applied directly over the entire tattooed area for a second specified period of time resulting in degradation of the tattoo ink.

### RELATED APPLICATIONS

This application is a continuation-in-part of co-pending U.S. application Ser. No. 12/381,134 filed on Mar. 6, 2009 entitled “ULTRA BRIGHT LED INDUCED TATTOO REMOVAL” and claims the priority of provisional application Ser. No. 61/068,369, filed Mar. 7, 2008, hereby incorporated by reference.

### BACKGROUND

A variety of medical procedures and techniques are currently being used to remove tattoos. For example, dermabrasion has been used to remove tattoos, in which the skin is simply sliced off or abraded. Dermabrasion has many adverse effects for one it may produce scars and, often, pigments which lie in different skin layers are not removed along with the others. In addition, a dark shade from the remnants of the tattoo may show through remains. Another method involves the tangential excision and covering with a “split-skin graft”. The skin layer concerned is cut out under general anesthesia, with as much as possible being saved from the underlying skin layer. The open area is covered with split skin, and saved from unnecessary scar formation over months by compression bandages, and adapted to the environment.

More recent methods of removing tattoos include the use of pulsed radiation. But these procedures and techniques may produce small volumes of relatively high power density that can cause significant local heating of tissues that may damage skin tissues. Also, these methods and procedures typically involve use of monochromatic light that may not be absorbed effectively by many dyes of varying colors. Laser treatment may also be used. This treatment entails delivering light energy to the tattoo in order to break the pigments into fragments, which are then removed by the subjects' immune system. The drawbacks to laser removal are that the majority of the power of the laser is wasted as heat that must be removed to prevent tissue damage. Also, these treatments can be very expensive, painful, and not always effective. In addition, using lasers can cause reactions in certain of the chemicals used in the inks, therefore, leading to permanent darkening. The known procedures for removing tattoos are expensive and not affordable to everyone. These procedures also cannot treat large surface areas and the treatment is focused on a very small area of a tattoo.

### SUMMARY OF THE DISCLOSED TECHNOLOGY

The disclosed technology relates a combination device for applying a treatment of light and ultrasound on a tattooed area of a subject for tattoo removal. The device includes an ultrasound source and a LED light panel that can be individually controlled.

The ultrasound device produces high-frequency ultrasound waves. The high frequency ultrasound waves have a frequency of about 5 Mhz (range 0.5-50) and an intensity of about 20 W/cm² (range 5-50). During treatment, the ultrasound sound waves can be administered over short times (5-30 seconds) in order to allow tissue recovery between each application. These waves are applied directly to the tattooed area for a specified period of time (approximately 10-15 minutes) resulting in cavitation of tattooed cell structures incorporating tattoo ink thus freeing the ink for subsequent disposal by the body.

The light panel houses a tight array of ultra-bright light emitting diodes (LEDs). The LEDs have a average wavelength between 400-700 nm resulting in (a) minimal absorption by melanin and hemoglobin of the subject and (b) little to no heat being generated on the epidermis of the subject while generating heat on the tattoo ink thereby causing increased molecular motion and bond deformation of the tattoo ink. The planar array of ultra-bright LEDs may be approximately equal in size to the tattooed area and has an energy output of about 50,000 Lux at typical treatment distances without the use of pulsed radiation. The LED produces a continuous light for that effect.

The light is directly applied over the entire tattooed area for a specified period of time (approximately 10-30 minutes) resulting in degradation of the tattoo ink and penetrates the epidermis of the subject without damaging the epidermis by overheating and it then enters the dermis of the subject in which tattoo ink resides.

### BRIEF DESCRIPTION OF DRAWINGS

FIG. 1 shows an overall view of apparatus used with this invention combining LED light and ultrasound.

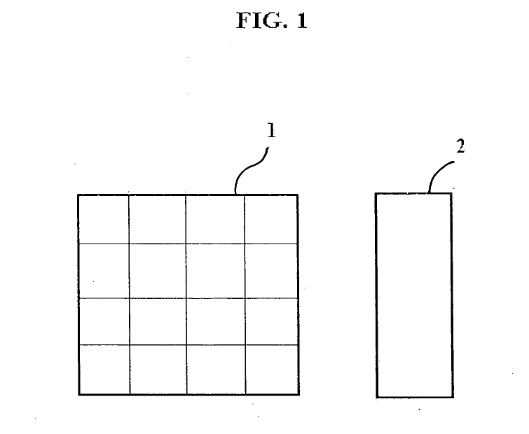

### DETAILED DESCRIPTION

Although specific terms are used in the following description for sake of clarity, these terms are intended to refer only to the particular structure of the invention selected for illustration in the drawings, and are not intended to define or limit the scope of the invention.

Following a tattoo application, dermal cells can consume and store tattoo particles in vacuoles or similar structures in the same manner fat cells store lipids. More specifically, tattoo ink contains carbon or inorganic particles that are suspended in water. When tattoo ink is introduced into the skin through a needle, the ink itself then spreads into the skin and can be absorbed by cells within the skin.

Primarily, three key parameters of ultrasound—frequency, intensity, and exposure time—play influential roles in the performance and efficacy of ultrasound-mediated therapies. When used as a tattoo removal technique it was found that high frequency ultrasound at certain intensities and application times can be used to target tattooed cells. In a preferred embodiment, an ultrasound device may use a high frequency ultrasound of 5 MHz and with a power of about 20 W/cm² delivered for about 10 minutes. Because continuous application of ultrasound can build up heat, the ultrasound can be delivered over discrete time intervals in order to allow tissue recovery between each interval.

When using ultrasound, regions of the tattooed cells may be selectively disrupted based on differences in mechanical and acoustic properties between ink particles and normal tissue. That is, different ultrasound frequencies and intensities may be used to free pigment particles of various sizes without damaging healthy tattoo-free tissue. The result is a technique that safely, economically, and efficiently removes at least significant portions of the ink. However, ultrasound alone may not remove all of the tattoo ink from the tattooed area.

It was found that if LED light was applied within a specified time after the application of ultrasound it would result inthe ink being more readily degraded and the body will more quickly rid itself of the tattoo ink. In use, it was also found that using certain wavelengths of light might disrupt the bonds that hold some types of tattoo ink together. In operation, the LED device works by using the energy contained in the light beam so that the energy is absorbed by the tattoo ink dyes. This absorbed energy can result in an increased stretching, vibration and bending of the bonds, which hold the dye (ink) molecules together. Ultimately, these bond stresses cause bond deformation with resulting bond failure.

The LED light frequencies chosen are those which produce energies which are absorbed by the bonds in the dyes but have minimal absorption by melanin in the skin or hemoglobin in the blood. Melanin and hemoglobin have maximum absorptions below 400 nm. Maximum absorption for melanin is 335 nm and for hemoglobin 310 nm. For the light produced to be beneficial for removal tattoos, ultra bright LEDs with high enough energy output are used. The output energy, such as will be approximately 50-100,000 Lux.

The control panel controls the plurality of ultra-bright LEDs and ultrasound. The ultrasound device produces high-frequency ultrasound waves. The high frequency ultrasound waves have a frequency of about 5 MHz and an intensity of about 20 W/cm2. The ultrasound sound waves are administered in pulses in order to allow tissue recovery between each pulse. These waves are applied directly to the tattooed area for a specified period of time (approximately 10 minutes).

The light panel houses a tight array of ultra-bright light emitting diodes (LEDs). The LEDs have an average wavelength between 400-700 nm resulting in (a) minimal absorption by melanin and hemoglobin of the subject and (b) little heat being generated on the epidermis of the subject while generating heat in the tattoo ink thereby causing increased molecular motion and bond deformation of the tattoo ink and produces a continuous light. The tight array of ultra-bright LEDs is approximately equal to size of the tattooed area and has an energy output of about 50.000 Lux without the use of pulsed radiation.

The light is directly applied over the entire tattooed area for a specified period of time (approximately 10-30 minutes) resulting in degradation of the tattoo ink and penetrates an epidermis of the subject without damaging the epidermis by overheating and enters the dermis of the subject in which tattoo ink resides.

In a preferred embodiment, the light panel includes a tight array of ultra-bright LEDs having an energy output of about 50,000 Lux without the use of pulsed radiation. The tight array of ultra-bright LEDs continuously applies the energy output from the tight array of ultra-bright LEDs directly over the entire tattooed area for a specified period of time resulting in degradation of the tattoo ink. Specifically, the optical device 1 has ultra bright LEDs. The ultra bright LEDs. The ultrasound unit is shown at 3

In use, L-Arginine can be applied to the tattooed region before administering the LED light. It creates enlarged blood vessels that bring greater blood flow to the tattoo area. In addition, it creates an increase in the immune system response. Additionally, an IRM (immune response modifier) compound can be applied. Specifically, IRM compounds containing L-Arginine can also increase the concentration of macrophages in the blood. Macrophages are specifically located in the lymph nodes and are white blood cells that phagocytizes necrotic cell debris and foreign material, including viruses, bacteria, and tattoo ink. These two mechanisms help speed up the removal of the by-products of the degradation of the tattoo dyes, thus, allowing for the tattoo to fade more quickly. The IRM compound may be selected from a group consisting of imidazoquinoline amine; a tetrahydroimidazoquinoline amine; an imidazopyridine amine; a 1,2-bridged imidazoquinoline amine; a 6,7-fused cycloalkylimidazopyridine amine; animidazonaphthyridine amine; a tetrahydronaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazodimer fused to a pyridine amine, a quinoline amine, a tetrahydroquinoline amine, a naphthyridine amine, and a tetrahydronaphthyridine amine.

### EXAMPLES

It will be understood that the following embodiments of the present invention are intended to be illustrative of some of the possible applications or principles. Various modifications may be made by the skilled person without departing from the true spirit and scope of the invention.

### Example

High frequency ultrasound having a frequency of 5 MHz and an intensity of 20 W/cm2 is applied for 10 minutes to a tattooed area treated with an ultrasound gel. After the ultrasound has been applied, the operator will wipe off the ultrasound gel, wait approximately two minutes for the patient's skin to recover, apply L-Arginine to the tattooed region and then place the LED apparatus approximately 1 to 2 inches above the tattooed area. The apparatus contains 16 ultra bright LEDs clustered in four rows of four LEDs each. The tattoo area is then exposed to the continuous light generated by the clustered ultra bright LEDs for 15 minutes. During this period of time, the light penetrates through the epidermis and into the dermal layer in which the tattoo ink resides. The absorption of the energy by the tattoo ink results in both heat generated in the ink particles and chemical dyes by molecular vibration and molecular bond deformation. This dual treatment is applied approximately six times over a few months.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. An apparatus for applying a treatment of light and ultrasound on a tattooed area of a subject for tattoo removal, the apparatus comprising:
an ultrasound device, the ultrasound device producing a high-frequency ultrasound waves, the ultrasound device applying the high-frequency ultrasound waves directly to the tattooed area for a first specified period of time; and
ultra-bright light emitting diodes (LEDs), for producing a continuous energy output directly over the entire tattooed area for a specified period of time resulting in degradation of the tattoo ink.

2. The apparatus of claim 1 wherein the light penetrates an epidermis of the subject without damaging the epidermis by overheating and enters a dermis of the subject in which tattoo ink resides.

3. The apparatus of Claim I wherein the LEDs have a average wavelength between 400-700 nm resulting in (a) minimal absorption by melanin and hemoglobin of the subject and (b) little to no heat being generated on the epidermis of the subject while generating heat in the tattoo ink.

4. The apparatus of claim 1 wherein the tight array of ultra-bright LEDs is approximately equal to size of the tattooed area.

5. The apparatus of claim 1 wherein the tight array of ultra-bright LEDs having an energy output of about 50,000 Lux without the use of pulsed radiation.

6. The apparatus of claim 1 wherein the second specified period of time is approximately 10-30 minutes.

7. The apparatus of claim 1 wherein the high frequency ultrasound device administers the ultrasound sound waves with a frequency of about 5 MHz and an intensity of about 20W/cm2.

8. The apparatus of claim 1 wherein the first specified period of time is approximately 10 minutes.

9. The apparatus of claim 1 wherein the ultrasound sound waves are administered in intervals in order to allow tissue recovery between each interval.

10. A method for removing tattoos comprising the steps irrespective of order:
applying an ultrasonic gel to a tattooed skin region;
positioning an ultrasonic device in direct contact with the tattooed area;
exposing the tattooed skin region to high-frequency ultrasound waves for a first specified period of time resulting in release of ink particles of tattooed cells;
positioning an optical device including a plurality of ultra bright LEDs at a specific distance from said tattooed skin region, and
exposing said tattooed skin region to continuous LED energy without pulsing in the range of 400 nm to 700 nm wavelengths for a timed interval.

11. A method in accordance with claim 10
with the addition of the step of applying L-argirine to a tattooed skin region.
